(19) United States
(12) Patent Application Publication
(10) Pub. No.: US 2025/0012743 A1
(43) Pub. Date: Jan. 9, 2025

(54) System and Method for Determining Properties of a Substrate

(71) Applicant: Eric Arno Vigen, Sherman, TX (US)

(72) Inventor: Eric Arno Vigen, Sherman, TX (US)

(21) Appl. No.: 18/675,016

(22) Filed: May 27, 2024

Related U.S. Application Data
(63) Continuation-in-part of application No. 18/375,768, filed on Oct. 2, 2023, now abandoned.
Application No. 18/375,768 is a continuation of application No. 17/139,271, filed on Dec. 31, 2020, now Pat. No. 11,774,382.
(60) Provisional application No. 63/468,822, filed on May 25, 2023.
(60) Provisional application No. 63/468,824, filed on May 25, 2023.
(60) Provisional application No. 63/468,827, filed on May 25, 2023.
(60) Provisional application No. 63/468,833, filed on May 25, 2023.
(60) Provisional application No. 63/468,838, filed on May 25, 2023.
(60) Provisional application No. 63/468,839, filed on May 25, 2023.
(60) Provisional application No. 63/652,079, filed on May 27, 2024.
(60) Provisional application No. 62/955,499, filed on Dec. 31, 2019.

(51) Int. Cl. G01N 23/2251; G01N 33/68; H01J 37/06; H01J 37/244

(52) U.S. Cl. CPC G01N 23/2251; G01N 33/6803; H01J 37/06; H01J 37/244; H01J 2237/2445

(57) ABSTRACT

A method and a system is disclosed in which a plurality of portions of a substrate are irradiated with a beam consisting essentially monochromatic physics spin isolated electromagnetic radiation to determine the atomic structure of the substrate based on a spectral line dataset.

### CROSS REFERENCE TO RELATED APPLICATIONS

The present application is a continuation-in-part, and claims priority to U.S. patent application Ser. No. 18/375,768, filed Oct. 2, 2023 now pending, which is a continuation of U.S. patent application Ser. No. 17/139,271, filed Dec. 31, 2020, now U.S. Pat. No. 11,774,382 which issued Oct. 3, 2023, which claims priority to U.S. Provisional Pat. App. No. 62/955,499, filed Dec. 31, 2019. The present application further claims priority to U.S. Provisional Pat. App. 63/468,822 filed May 25, 2023; and to U.S. Provisional Pat. App. 63/468,824 filed May 25, 2023; and to U.S. Provisional Pat. App. 63/468,827 filed May 25, 2023; and to U.S. Provisional Pat. App. 63/468,833 filed May 25, 2023; and to U.S. Provisional Pat. App. 63/468,838 filed May 25, 2023; and to U.S. Provisional Pat. App. 63/468,839 filed May 25, 2023; and to U.S. Provisional Pat. App. 63/652,079 filed May 27, 2024. The disclosures of all the above patents, patent applications, and provisional patent applications are hereby incorporated herein by reference.

### FIELD

Embodiments of the present disclosure generally relate to methods and system for analysis of a substrate. More specifically, to a system for examination and re-examination of materials for identification of the material composition. In particular, methods and systems using physics-spin-isolated electromagnetic radiation to generate photons from a substrate, wherein the generated photons are analyzed for information, examination, and reexamination relating to a composition and arrangement of the material.

### BACKGROUND

There is a need in the art to determine atomic composition and atomic arrangement of substances. Materials irradiated with electromagnetic energy e.g., light, emit photons of having a wavelength, frequency, and energy related to a composition of the material. However, current analysis techniques currently lack the precision to discern particle interactions based on an arrangement and composition of the material. There is an inverse relationship between those two attributes (frequency and energy) and the opposition factor of distance precision. The relationship between the two attributes of higher frequency and energy, and the opposing factor of measurement precision is more closely related to lower wavelengths.

It is well established and understood that higher frequency, for smaller wavelengths, for greater precision, requires much greater energy, and has other risks, like X-rays destroy the tissues of living beings. As such, the present invention is a method to use that dataset from a better, safer frequency level and strength, and thereby energy, lower in the scale, yet achieve precision at a higher level. That is useful to get smaller distance measurement without the excessive energy and its risks of extremely small wavelengths which require extremely high energy.

Accordingly, there is a need for determining the composition and arrangement of substrates utilizing low energy sources.

### SUMMARY

In embodiments, a method to determine an atomic structure of a substrate comprises irradiating a plurality of portions of the substrate with a beam of essentially monochromatic physics spin isolated electromagnetic radiation having a beam wavelength λ_{EM} oriented in a beam direction, the beam contacting the substrate at an angle of incidence relative to the substrate, for a period of time sufficient to produce an emission of photons from each of the plurality of portions forming one or more emission spectral lines corresponding to a particular portion of the substrate, wherein the emission photons emanate from the substrate at an emission angle which is different from the angle of incidence; analyzing the emission spectral lines produced by the photons from each of the plurality of portions to produce an emission spectral line dataset, comprising: an emission angle of the emission photons which formed the corresponding spectral line relative to the beam direction; a wavelength of each corresponding spectral line λ_{sub}, and/or a polarity of the photons forming the corresponding spectral line; and determining the atomic structure of the substrate based at least in part on the spectral line dataset.

In embodiments, a system for determining an atomic structure of a substrate comprises a source of a beam of monochromatic physics spin isolated electromagnetic radiation having a beam wavelength λ_{EM}, an irradiation system configured to irradiate a plurality of portions of a substrate with the beam of monochromatic physics spin isolated electromagnetic radiation in a beam direction such that the beam contacts the substrate at an angle of incidence relative to the substrate for a period of time sufficient to produce an emission of photons from each of the plurality of portions forming one or more emission spectral lines corresponding to a particular portion of the substrate, an analysis system configured to analyze the emission photons emanating from the substrate at an emission angle which is different from the angle of incidence; the analyzing system configured to determine the emission spectral lines produced by the photons from each of the plurality of portions to produce an emission spectral line dataset comprising: an emission angle of the emission photons which formed the corresponding spectral line relative to the beam direction; a wavelength of each corresponding spectral line λ_{sub}, and/or a polarity of the photons forming the corresponding spectral line; and determining the atomic structure of the substrate based at least in part on the spectral line dataset.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a flow chart depicting a method according to embodiments disclosed herein.

FIG. 2 depicts electron shell arrangement using the HemiChem model according to embodiments disclosed herein.

FIG. 3 depicts a system for determining an atomic structure of a substrate according to embodiments disclosed herein.

FIG. 4 depicts a portion of the system shown in FIG. 3.

FIG. 5 depicts a physics spin isolated electromagnetic energy beam according to embodiments disclosed herein.

FIG. 6 depicts a modified Aharonov Bohm filter to produce a physics spin isolated electromagnetic energy beam according to embodiments disclosed herein.

FIG. 7 depicts a modified double/single slit filter to produce a physics spin isolated electromagnetic energy beam according to embodiments disclosed herein.

FIG. 8 depicts a modified Stern Gerlach filter to produce a physics spin isolated electromagnetic energy beam according to embodiments disclosed herein.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Embodiments disclosed herein include reference to a hemispherical model, referred to herein as a Hemichem model, which provides physical modelling of the underlying chemistry and subatomic physics using hemispherical coordinates (r_{z},θ_{z},a_{z},z_{z}=X0=±½), replacing quantum numbers (n,l,mₗ,mₛ). The hemispherical model further utilizes acceleration allocation (aa) always at the system center-of-mass, thereby replacing Newton's 2nd Law of Motion wherein three core interactions are utilized to generate the fundamental forces, including full integration of gravity.

For purposes herein, elementary particles are considered to be elementary-event sets of multiple core particles, arranged in three dimensional (3D) engineering stable structures. The Hemichem model provides 3D engineering physical arrangement, along with causation of quantum equations in terms of classical physical understanding and calculations. Current quantum mechanical analysis is based on statistical techniques which remain valid for multiple particles and multiple events. However, current quantum mechanical analysis cannot describe a single event or particle. In contrast, the Hemichem model utilized herein provides a means of obtaining information in terms of 3D engineering of quantum equations known in the art by incorporating an additional frame-of-reference. Various known dilemmas, such as improper infinities and the like are resolved by the Hemichem model, rendering statistical quantum techniques redundant.

However, the Hemichem method referred to herein does not detract from, and is in overall agreement with the multitude of information readily known to one of skill in the art of quantum mechanics (QM), quantum field theory (QFT), and the like. The Hemichem method utilized herein presents revisions to known relationships which bridge classical single particle-particle events-sets with the multiple-event quantum prediction techniques and experimental evidence.

A detailed description of the methods and models utilized herein, including the HemiChem model and the application thereof, may be found in the HemiChem IDS series including Vigen, A. (Apr. 17, 2024). Hemispherical Atomic Model ISBN 9798870681290, Vigen, A. (Dec. 23, 2023). The Nature and Causation of Light, Photons, and EM Waves Amazon-ASIN B0CQWN1NT4; Vigen, A. (3-1-2024). First Principles for Subatomic Physics Amazon-ASIN: B0CWTYVX7Q; and the like. Additional references include Vigen, A. (Jan. 28, 2023). HemiQuantum Physics: Resolving Each Quantum Dilemma: Improving Each Quantum Technique from Planck's Equation to Elementary Particles, Amazon-ASIN B0BTC7PGND; Vigen, A. (Jan. 19, 2019). Understanding Pauli's ½ as 3D Hemispheres Fully Links Quantum Theory to Classical Physics, Amazon-ASIN B07MYNTPJ7; Vigen, A. (Jan. 18, 2019). Simple Words to Fully Reconcile Classical Mechanics with Quantum Theory, Amazon-ASIN B07MY9CH4F; Vigen, A. (Apr. 25, 2022).Refining the Schrödinger Wave Function Equation in Hemispherical (r,θ,φ,z=X0=±½) Coordinates Amazon-ASIN B09YWSL1FR; Vigen, A (Oct. 12, 2016).Gravity is Just That Electrons are a Little Closer; Amazon-ASIN B01M9B4V4E; Vigen. A (Oct. 13, 2016). Electron Shell Chemistry is Just Scrunched Cube Geometry; Amazon-ASIN B01M7PRGWZ; Vigen. A (Jan. 19, 2019).Revising Planck-Einstein Energy Equation to Add Pauli's ½ Fully Links Quantum Theory to Classical Physics; Amazon-ASIN (B07MYLTJ67); Vigen. A (Jan. 18, 2018). Postulated Nucleostaticmagnetics Force for Subatomic Particles Resolves Dirac's 1931 Monopoles as Fully Deterministic Duopoles; Amazon-ASIN B07MY7F289; Vigen. A (Mar. 25, 2023). The Mass Equation: My Breakthrough Position-in-Field Approach from Hemispherical (r,θ,ϕ,z=X0=±½); Amazon-ASIN B0BZN3MGNC; Vigen A. (Oct. 2, 2020). Replacement of Bohr's Angular Momentum with Strong Nuclear Force for Electrons; Amazon-ASIN B08KNMVPB1; Vigen, A (Jun. 20, 2020). Nucleostaticmagnetics Vector Equations; Amazon-ASIN B08BKW46GG; Vigen, A. (Mar. 2, 2020). Math Integrity Understanding Strong and Weak Force Through the Forces/Fields of Electrostatic, Direct and Axial Nucleostaticmagnetics: 4-Vector in 3D Model Generating Four Quantum Equations (Dirac); Amazon-ASIN B085R99M44; Vigen, A. (Jan. 18, 2020). Renaissance Physics: Understanding Post-Quantum Novo-Classical Subatomic Particle Engineering Textbook Chapter 1-4 Amazon ISBN 1659185777; Vigen, A. (Mar. 31, 2019). 3D Visual Chemistry Textbook; Amazon-ASIN B07Q3PY8GV; Vigen, A. (Jan. 18, 2018). Quantum Entanglement, Wave Functions, and Spectrum Given the 3D Arno Vigen Scrunched Cube (AVSC) Atomic Model; Amazon-ASIN B07MY7Y5ZW; Vigen, A. (Oct. 8, 2017). Fixing Einstein's E=mc-squared: Replacing Observed Mass (‘m’) with the ‘M’ Nucleus Magnetic Force Divided by the Volume of the Electron Shell Radius Separation; Amazon-ASIN B0769ZJK9K; Vigen, A. (Oct. 29, 2016). Why Does a Nucleus Stay Together When Protons (+) Repel Each Other?: A Nucleus is Just . . . a Magnetic Chain-Ring; Amazon-ASIN B01M73KXNQ; The full disclosures of each are fully incorporated by reference herein.

As used herein, radial electrostatic (rES) interaction/force refers to an electrostatic (ES) attribute often referred to as charge in the prior art, having an appropriate sign of positive (+) or negative (−) or zero for neutrons. The operating rules associated with radial electrostatic (rES) interaction is that opposites attract and like-kind repel with no interaction when the value is zero (0).

Xtrastatic (XS) axis refers to the magnetic axis inherent in every subatomic particle according to the Hemichem model. Axial xtrastatic interaction and/or force refers to the attractive force from a particle (P1), or more specifically from its two hemispheres/poles, towards the axis of a second particle (P2). The sign of the interaction being based upon the XS-attribute known as mass in prior art. The operating rules associated with xtrastatic (XS) interaction as like kind zero and difference generating two force vectors rXS isotropic repulsive with aXS as anisotropic attribute to the axis of the other particle-set. These also split between portions as linear towards-the-axis and portions as rotational of the axis itself.

Radial xtrastatic interaction and/or force refers to the repulsive force from a particle (P1) from its two differentiated axis/hemispheres away from a second particle (P2). This sign of this interaction is based upon the XS-attribute also referred to as mass according to common understanding in the art.

In addition, it is understood that like-kind particles do not have xtrastatic interactions. Only the electron-proton, and the electron-nucleon have xtrastatic interactions.

For purposes herein, consistent with the Hemichem model, particle-edge and maximum field strength occurs at a particle's physical dimension radius, which corresponds to the Bohr radius, abbreviated herein as (re). Accordingly, for purposes herein, a proton is assumed to have a radius and position-in-field maximum at (re).

For purposes herein, calculation of the behavior of each hemisphere is defined from a pole to an equator of the hemisphere by the inherent XS axis over the body of the particle. The body of the particle having a center-of-substance defined in hemispherical coordinates (r_{z},θ_{z}, φ_{z},z_{z}=X0=±½) as ((⅜)r_{z},0,0,+½) for a first hemisphere, and ((⅜)r_{z},0,0,−½) for the other second hemisphere, which is locked-at-180° relative to the first hemisphere.

As used herein, radial electrostatic force—(rES)—refers to the interaction between protons and electron based upon the charge attribute with the product as the interactions. Accordingly, “opposites attract” and “like-king repel” based upon the following table wherein neutrons do not experience interactions with either a proton or an electron.

Table 1 depicts the interactions for subatomic particles for radial electrostatic (rES) interactions.

For the radial electrostatic (rES) interactions, the logic table for types is based upon the product of the signs. The rES ‘charge’ attributes are as follows:
Proton=(+1); Neutrons=0; and Electrons=(−1); which are shown in the Table 1:

TABLE 1
OF ELECTROSTATIC CHARGE INTERACTIONS

| | Proton (+) | Neutron (0) | Electrons (−) |
|---|---|---|---|
| PROTON | (+)(+) = (+) | (0)(+) = (0) | (+)(−) = (−) |
| NEUTRON | (+)(0) = (0) | (0)(0) = (0) | (−)(0) = (0) |
| ELECTRONS | (+)(−) = (−) | (0)(−) = (0) | (−)(−) = (+) |

wherein the xtrastatic, pre-magnetism subatomic force, the interaction logic table is based upon two force vectors:
i) Radial xtrastatic, the outward ‘at the equator; and
ii) Axial xtrastatic, the inward towards-the-axis.

These operate by ‘like-kind zero’ and both operating in different directions with an overall sign opposite to rES. That is the formula is (SIGN) (product) wherein both protons and neutrons have this ‘mass’ attribute as (+1).

For the XS ‘mass’ attribute the assigned attributes are:
Proton=(+1), Neutrons=(+1) and Electrons=(−1), wherein the radial xtrastatic (rXS), using sign=(−1), is shown in Table 2:

TABLE 2
OF RADIAL XTRASTATIC CHARGE INTERACTIONS

| | Proton (+) | Neutron (0) | Electrons (−) |
|---|---|---|---|
| PROTON | same = (0) | same = (0) | (−)((+)(−)) = (+) |
| NEUTRON | same = (0) | same = (0) | (−)((+)(−)) = (+) |
| ELECTRONS | (−)((+)(−)) = (+) | (−)((+)(−)) = (+) | same = (0) |

Axial xtrastatic (aXR) (using sign=(+1)) are shown in Table 3:

TABLE 3
OF AXIAL XTRASTATIC CHARGE INTERACTIONS

| | Proton (+) | Neutron (0) | Electrons (−) |
|---|---|---|---|
| PROTON | same = (0) | same = (0) | (+)((+)(−)) = (−) |
| NEUTRON | same = (0) | same = (0) | (+)((+)(−)) = (−) |
| ELECTRONS | (+)((+)(−)) = (−) | (+)((+)(−)) = (−) | same = (0) |

The combination two static force vectors of the axial xtrastatic force is out-at-equator, inward-towards-axis of pre-magnetism, and the electron subshells, which relates to the inclination ring-spring physics underlying Bose proof of quantum mechanics (QM).

Elemental Ionization Energy—(Ei,N) refers to the energy to remove an electron of a chosen element. For example, the energy required to remove an electron from a hydrogen atom to produce a proton, wherein N=1 for hydrogen.

Molecular Ionization Energy—(Ei,AB−) refers to the energy to remove an electron of a chosen molecular state. For example, the energy required to ionize water to produce a hydronium or hydrogen ion (H₂O)⁺. In this example, the AB notation above is H₂O.

As used herein, a physics spin isolated electromagnetic energy beam, also referred to simply as a spin isolated electromagnetic energy beam, refers to a monochromatic electromagnetic beam consisting essentially of a plurality of physics spin isolated photons, wherein the physics spin isolated photons are only observable within a plurality of first discrete ranges along a path of the monochromatic electromagnetic beam, each of the first discrete ranges centered at a corresponding distance from a source of the monochromatic electromagnetic beam, wherein essentially no photons are observable within a plurality of second discrete ranges located in-between each of the first discrete ranges, as described in the Applicant's corresponding U.S. patent application Ser. No. 18/674,995, filed May 27, 2024, the disclosure of which is incorporated by reference herein.

In embodiments, a method to determine an atomic structure of a substrate, comprises irradiating a plurality of portions of the substrate with a beam of essentially monochromatic physics spin isolated electromagnetic radiation having a beam wavelength λ_{EM} oriented in a beam direction, the beam contacting the substrate at an angle of incidence relative to the substrate, for a period of time sufficient to produce an emission of photons from each of the plurality of portions forming one or more emission spectral lines corresponding to a particular portion of the substrate, wherein the emission photons emanate from the substrate at an emission angle which is different from the angle of incidence; analyzing the emission spectral lines produced by the photons from each of the plurality of portions to produce an emission spectral line dataset, comprising: an emission angle of the emission photons which formed the corresponding spectral line relative to the beam direction; a wavelength of each corresponding spectral line λ_{sub}, and/or a polarity of the photons forming the corresponding spectral line; and determining the atomic structure of the substrate based at least in part on the spectral line dataset.

In some embodiments, the determining of the atomic structure comprises determining an arrangement of subatomic particles and the subatomic particles present at each of the plurality of portions based at least in part on the corresponding spectral line dataset, wherein a distance between a nucleus and an electron of an atom of element E which produced the emission spectral line is determined according to formula (I):

$${λ}_{sub} = {R}_{E , ⊖ \#} ( 1 / {( {N}_{1} )}^{2} - 1 / {( {N}_{2} )}^{2} ) ; \qquad ( I )$$

wherein λ_{sub} is a wavelength of the emission spectral line; θ# is an integer second quantum number of the subshell of the electron of the atom which produced the emission spectral line; R_{E,θ#} is a Rydberg constant for the subshell of the electron of the atom of element E which produced the emission spectral line; Nₓ is an integer subset energy level of the element E of the atom which produced the emission spectral line, starting at x=1, determined by formula (II)

$${N}_{x + 1} = {N}_{x} + 1 ; \qquad ( II )$$

wherein each N and x are determined independently, as integers greater than or equal to 1; and wherein R_{E,θ#} is determined by formula (III):

$${R}_{E , ⊖ \#} = 6 {r}_{e} ( {( {D}_{eN} / ( 6 {r}_{e} ) )}^{2} - 1 ) ; \qquad ( III )$$

wherein: rₑ is the radius of an electron; and D_{eN} is the distance between the nucleus and the electron of the atom of the element E which produced the emission spectral line. In embodiments, the determining of the atomic structure further comprises determining an emission angle between an xtrastatic axis of the nucleus and the electron of the atom of the substrate which produced the emission spectral line, wherein the nucleus is a vertex of the angle, according to formula (IV):

$$cos ( {⊖}_{emission} ) = \frac{1}{( 2 * ( r \# ) - 1 )} \qquad ( IV )$$

wherein θₑₘᵢₛₛᵢₒₙ is the emission angle in radians, ±0.1 radians, of the electron relative to the xtrastatic axis of the nucleus and the electron of the atom of the substrate which produced the emission spectral line, with the nucleus as the vertex for the atomic structure of the substrate; θ# is an integer second quantum number of the subshell of the electron of the atom which produced the emission spectral line; and r # is the integer first quantum number of the shell of the electron of the atom which produced the emission spectral line.

In some embodiments, the determining of the atomic structure further comprises determining an electron arrangement of the atom of the substrate which produced the emission spectral line; wherein a direction of covalent bonding between the atom of the substrate which produced the emission spectral line and another atom present within the substrate is based at least in part on a predetermined data set comprising a plurality of predetermined distances between a nucleus and an electron of an atom of element E, and a plurality of predetermined emission angles between bonding electrons and a corresponding nucleus of an atom of element E, wherein: the predetermined data set includes only full subshells sets; and the subshell sets are selected from one or more of: Subshell-s having 2 electrons; Subshell-p having 6 electrons; Subshell-d having 10 electrons; Subshell-f having 14 electrons; and/or equatorial (TT radian) sets having 3, 5, or 7 electrons each.

In embodiments, the method further comprises changing a distance between a source of the beam of monochromatic physics spin isolated electromagnetic radiation and the substrate over a range configured to irradiate the portions of the substrate at intervals of greater than or equal to about ⅜ rₑ, wherein rₑ is the radius of an electron equal to about 2.8179*10⁻¹⁵ m.

In embodiments, the irradiating of the plurality of portions of the substrate comprises a plurality of irradiations of the same substrate, each utilizing a different beam wavelength λ_{EM}; a different beam direction; a different angle of incidence; or a combination thereof.

In embodiments, the substrate comprises a protein sequence. In embodiments, the substrate comprises a semi-conductor substrate. In embodiments, the atomic structure of at least one portion of the substrate is dependent on an occurrence or non-occurrence for a computer operation, e.g., a quantum computer. In embodiments, the computer operation comprises a change in electron energy level of an electronic memory of a computer.

In embodiments, a system for determining an atomic structure of a substrate, comprises a source of a beam of monochromatic physics spin isolated electromagnetic radiation having a beam wavelength λ_{EM}; an irradiation system configured to irradiate a plurality of portions of a substrate with the beam of monochromatic physics spin isolated electromagnetic radiation in a beam direction such that the beam contacts the substrate at an angle of incidence relative to the substrate for a period of time sufficient to produce an emission of photons from each of the plurality of portions forming one or more emission spectral lines corresponding to a particular portion of the substrate; an analysis system configured to analyze the emission photons emanating from the substrate at an emission angle which is different from the angle of incidence; the analyzing system configured to determine the emission spectral lines produced by the photons from each of the plurality of portions to produce an emission spectral line dataset comprising: an emission angle of the emission photons which formed the corresponding spectral line relative to the beam direction; a wavelength of each corresponding spectral line λ_{sub}, and/or a polarity of the photons forming the corresponding spectral line; and determining the atomic structure of the substrate based at least in part on the spectral line dataset.

In embodiments the system is further configured to change a distance between the source of the beam of monochromatic physics spin isolated electromagnetic radiation and the substrate over a range configured to irradiate the portions of the substrate at intervals of greater than or equal to about ⅜ rₑ, wherein rₑ is the radius of an electron equal to about 2.8179*10⁻¹⁵ m.

In embodiments the system is further configured to change the beam wavelength λ_{EM}; the beam direction; the angle of incidence; or a combination thereof.

In embodiments the system is further configured to conduct a plurality of irradiations on the same substrate, each utilizing a different beam wavelength λ_{EM}; a different beam direction; a different angle of incidence; or a combination thereof.

In embodiments, the atomic structure of at least one portion of the substrate is dependent on an occurrence or non-occurrence for a computer operation. In some embodiments, the computer operation comprises a change in electron energy level of an electronic memory of a computer.

Light, specifically each photon, becomes an information set which generate models of molecules that include 3D engineering positions for its constituent subatomic particles (protons, electrons, and neutrons), within a few electron-diameter tolerance a low temperatures, of each electrons and nucleus set of such molecule, that react with mechanical adjustments, and thereby change the particles, energy levels. This engineering in present invention teaching allow methods and uses that take the subatomic particles a) energy levels, b) positions, c) polarity and spin, which determine d) bonding strength into models which then generate further actions, which has use in i) manufacturing, and ii) information systems, e.g. computer systems.

Light is powerful information. It tells the data for interactions too small for us to observe. The bright green of spring indicates new chlorophyll has been activated in plants without the precision of our unaided vision to discern that cell and the molecules. The unaided eye cannot observe a PS-I site in the thylakoid wall. However, the interaction of molecular bonds specific to the knowable interaction of the particles in combinations of Elements of the Periodic Table creates each set of specific color frequencies and wavelengths. The abundance of specific colors tells the macro-world about the micro-world. All this is true without microscopes, yet prior art and the present invention imaging and microscopy methods and systems generate more understanding beyond just the overall information of human vision, which operates by the general abundance (many green photons) of certain wavelength photons, based upon visible light, or more technically various types electromagnetic waves. There are prior art and present invention techniques to discern, gather and use more and better information, particularly tiny, subatomic dimension information of inner layer molecules as determined by the light of larger dimensions (that is lower-energy, longer-wavelength), and from that thereby to generate more precise methods for further actions.

In embodiments, the system disclosed herein utilizes lower energy, and thereby lower risk and lower cost, examination methods at the molecular and subatomic particle level. The system of the present invention gathers and validates information about molecules first as a) position, then as b) orientation, and finally as c) polarity relative to internal axis of the nucleus.

The present disclosure is directed to an apparatus and system combining causation of photon emission by low-energy electrostatic methods via knowable paths along, or even through. the target material. Capture and measurement of photons based upon direction, wavelength and/or polarity according to embodiments disclosed herein results in a data set suitable to determine the smaller internal molecular distance of an electron-nucleus-electron set consistent and stable for understanding the molecular structure within a range square root the distance of the wavelength data. That translation solves the precision-energy dilemma of prior art. In embodiments, precision while using lower energy than prior art is obtained which is utilized to model a target object molecular composition based upon the 3D Hemichem model electron-nucleus-electron sets.

Manipulation for either or both causation and capture/measurement based upon the model and its predictions. That is a mechanical process integrated with a computerized determine management of current directions, receptors, and potentially the object not possible without the process of examine-model-re-examine according to embodiments disclosed herein.

For purposes herein, the general Rydberg constant is represented as R∞, wherein R∞=10,973,731.57 m⁻¹.

The general Rydberg constant is calculated according to formula (I):

$${R}_{∞} = ( {( \frac{( \frac{{a}_{0}}{6 {r}_{e}} )}{{( 1 + {a}_{e} )}^{\frac{( \frac{3}{2} + 2 )}{2}}} )}^{2} ( 1 + \frac{1}{{2}^{3}} ) ) = {( \frac{( \frac{{a}_{0}}{6 {r}_{e}} )}{{( 1.0011589 )}^{\frac{7}{4}}} )}^{2} ( \frac{9}{8} ) \qquad ( I )$$

wherein a₀ is the Bohr hydrogen radius 5.2918*10⁻¹¹ m; rₑ is the radius of electron 2.818*10⁻¹⁵ m; and ae is the electron magnetic moment of 0.0011589, also referred to in the art as the anomalous moment.

The instant disclosure is directed to calculation of the translation from wavelength to electron-nucleon using 3D engineering radial and direct distance according to the HemiChem model.

In embodiments, irradiation of a substrate with the beam of essentially monochromatic physics spin isolated electromagnetic radiation results in a photon being emitted from a substrate due to the interaction of the beam with a nucleon or nucleus-set and a corresponding electron. The emission of the substrate occurs at angles other than the angle of incidence of the beam used to irradiate the substrate. Accordingly, embodiments disclosed herein utilize emission or scintillation of the substrate and do not require, or utilize a comparison in absorption between incident electromagnetic radiation and reflected electromagnetic radiation.

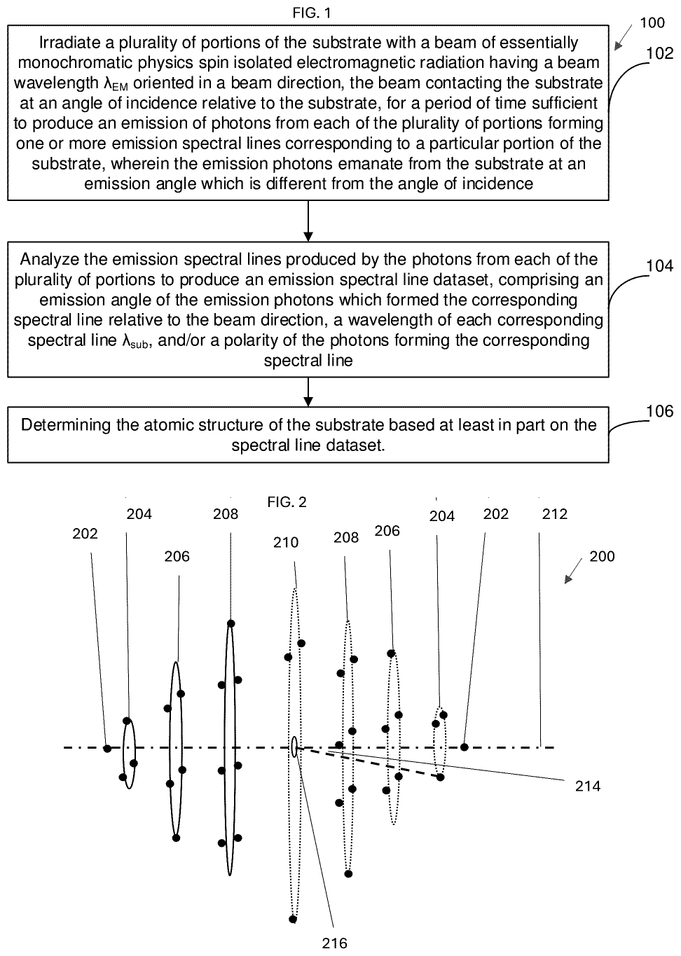

FIG. 1 depicts a block diagram of a method to determine an atomic structure of a substrate 100 according to the instant disclosure. In embodiments, the method includes irradiating a plurality of portions of the substrate with a beam of essentially monochromatic physics spin isolated electromagnetic radiation having a beam wavelength λ_{EM} oriented in a beam direction, the beam contacting the substrate at an angle of incidence relative to the substrate, for a period of time sufficient to produce an emission of photons from each of the plurality of portions forming one or more emission spectral lines corresponding to a particular portion of the substrate, wherein the emission photons emanate from the substrate at an emission angle which is different from the angle of incidence (block 102), followed by analyzing the emission spectral lines produced by the photons from each of the plurality of portions to produce an emission spectral line dataset, comprising an emission angle of the emission photons which formed the corresponding spectral line relative to the beam direction; a wavelength of each corresponding spectral line λ_{sub}, and/or a polarity of the photons forming the corresponding spectral line (block 104). The atomic structure of the substrate is then determined based at least in part on the spectral line dataset (block 106).

The Hemichem model is utilized to determine the atomic structure of the substrate. In embodiments, a distance between a nucleus and an electron of an atom of element E which produced an emission spectral line of the emission spectral line dataset for the substrate is determined according to formula (I):

$${λ}_{sub} = {R}_{E , ⊖ \#} ( 1 / {( {N}_{1} )}^{2} - 1 / {( {N}_{2} )}^{2} ) ; \qquad ( I )$$

wherein:
λ_{sub} is a wavelength of the emission spectral line;
θ# is an integer second quantum number of the subshell of the electron of the atom which produced the emission spectral line;
R_{E,θ#} is a Rydberg constant for the subshell of the electron of the atom of element E which produced the emission spectral line;
Nₓ is an integer subset energy level of the element E of the atom which produced the emission spectral line, starting at x=1, determined by formula (II)

$${N}_{x + 1} = {N}_{x} + 1 ; \qquad ( II )$$

wherein each N and x are determined independently, as integers greater than or equal to 1; and
wherein R_{E,θ#} is determined by formula (III):

$${R}_{E , ⊖ \#} = 6 {r}_{e} ( {( {D}_{eN} / ( 6 {r}_{e} ) )}^{2} - 1 ) ; \qquad ( III )$$

wherein:
rₑ is the radius of an electron; and
D_{eN} is the distance between the nucleus and the electron of the atom of the element E which produced the emission spectral line.

The emission angle associated with the emission spectral line dataset for the substrate, which represents the angle between an xtrastatic axis of the nucleus according to the Hemichem model, and the electron of the atom of the substrate, wherein the nucleus of the atom is a vertex of the angle, is determined from the emission spectral line dataset according to formula (IV):

$$cos ( {⊖}_{emission} ) = \frac{1}{( 2 * ( r \# ) - 1 )} ( \frac{π}{2} ) \qquad ( IV )$$

wherein:
θₑₘᵢₛₛᵢₒₙ is the emission angle in radians, ±0.1 radians, of the electron relative to the xtrastatic axis of the nucleus and the electron of the atom of the substrate which produced the emission spectral line, with the nucleus as the vertex for the atomic structure of the substrate;
θ# is an integer second quantum number of the subshell of the electron of the atom which produced the emission spectral line; and
r # is the integer first quantum number of the shell of the electron of the atom which produced the emission spectral line.

In embodiments, the determination of the atomic structure of the substrate is based at least in part on a predetermined data set comprising a plurality of predetermined distances between a nucleus and an electron of an atom of element E, and a plurality of predetermined emission angles between bonding electrons and a corresponding nucleus of an atom of element E. In embodiments, this predetermined data set includes data obtained from analysis of control or known materials, as well as theoretically derived data.

As shown in FIG. 2, the predetermined data set includes only full subshells sets 200; wherein the subshell sets are selected from one or more of:
Subshell-s (202) having 2 electrons;
Subshell-p (204) having 6 electrons;
Subshell-d (206) having 10 electrons;
Subshell-f (208) having 14 electrons; and/or
Equatorial (π radian) electron shells having 3, 5, or 7 electrons each (210).

The set of electrons 200 depicted in FIG. 2 are full sets from the ring-spring subshells sets perpendicular to the axis of the nucleus 216. For the xtrastatic axis (212) of the nucleus, the electron subshells are shown in 2 hemispheres along the xtrastatic axis 212, wherein the Subshell s electrons 202 have a first quantum number equal to 0, the Subshell-p electrons 204 of the 2 hemispheres ring-spring set of 3 each have a first quantum number of 1; the Subshell-d electrons 206 of the 2 hemispheres ring-spring set of 5 each have a first quantum number of 2; the Subshell-f electrons (208) of the 2 hemispheres ring-spring set of 7 each have a first quantum number of 3; and the equatorial Subshell-eq (210) of 1 ring-spring set at the equator relative to the xtrastatic axis 212 of the nucleus 216 have 3, 5, or 7 electrons. Accordingly, a subshell-s electron (202) has an angle of zero wherein the nucleus (216) is the vertex relative to the xtrastatic axis (212). The Subshell-p electrons 204 have an angle 214 wherein the nucleus (216) is the vertex relative to the xtrastatic axis (212).

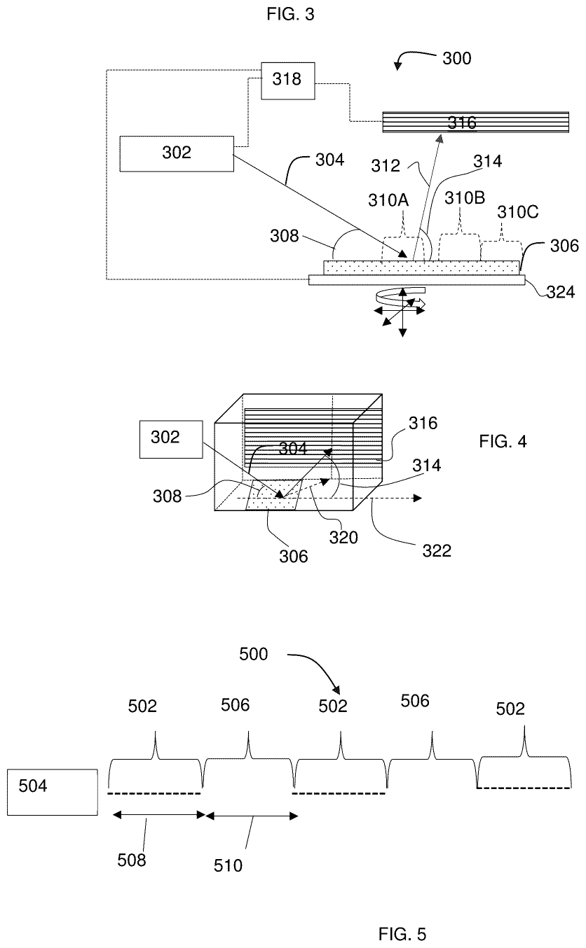

FIG. 3 depicts a block diagram of a system 300 for determining an atomic structure of a substrate according to embodiments disclosed herein. In embodiments, the system 300 includes an irradiation system 302 which produces a beam 304 of monochromatic physics spin isolated electromagnetic radiation having a beam wavelength λ_{EM}. The irradiation system 302 is dimensioned, configured and arranged such that the beam 304 oriented in a beam direction 322 (see FIG. 4) contacts a substrate 306 at an angle of incidence 308 to a surface of the substrate 306. The irradiation system is further configured to irradiate a plurality of portions 310A, 310B, 310C of the substrate 306 with the beam 304 of monochromatic physics spin isolated electromagnetic radiation. In embodiments, the substrate 306 is engaged on a movable stage 324, which allows the substrate 306 to be moved in any x, y, and z direction, and/or rotated. The beam 304 contacts the substrate 306 at the angle of incidence 308 at an intensity and for a period of time sufficient to produce an emission of photons 312 from each of the plurality of portions 310A. The emission photons 312, emanate from the substrate 306 at an emission angle 314 which is different from the angle of incidence 308, i.e., are not merely reflected from the beam 304, and in an emission direction 320 (See FIG. 4) relative to beam direction 322 and are detected by detection system 316, which is in electrical communication with a controller 318 which in embodiments is configured to analyze the emission spectral lines produced by the emission photons 312 from each of the plurality of portions 310, to produce an emission spectral line dataset, comprising an emission angle of the emission photons which formed the corresponding spectral line relative to the beam direction, a wavelength of each corresponding spectral line λ_{sub}, and/or a polarity of the photons forming the corresponding spectral line; and determining the atomic structure of the substrate based at least in part on the spectral line dataset. In embodiments, the controller 318 is configured to control the movable stage 324, and/or the irradiation system 302.

In embodiments, the system for determining an atomic structure of a substrate may include a plurality of sensors, detectors, and/or the like, for receiving and measuring the emission photons to generate the emission spectral line dataset comprising an emission angle of the emission photons which formed the corresponding spectral line relative to the beam direction, a wavelength of each corresponding spectral line λ_{sub}, and/or a polarity of the photons forming the corresponding spectral line of direction, wavelength, and polarity.

The emission spectral line dataset can further be defined as the photon direction as a line defining the geometric engineering of the one or more sets of electron-nucleus electrons; the photon wavelength determines the 3D internal dimension of one of the one or more sets of electron-nucleus electrons; and the photon polarity as a 3D rotational direction relative to the one or more sets of electron-nucleus electrons, including subshell-s.

In embodiments, the controller may be used to receive the data set of information for analysis, and includes a computer, along with the platform and associated software, configured to generate the emission spectral line dataset, and determine the atomic structure of the substrate based on the Hemichem model wherein a direction of covalent bonding between the atom of the substrate which produced the emission spectral line and another atom present within the substrate is based at least in part on a predetermined data set comprising a plurality of predetermined distances between a nucleus and an electron of an atom of element E, and a plurality of predetermined emission angles between bonding electrons and a corresponding nucleus of an atom of element E.

The model being a model of subatomic particle placement for the one or more photons by one or more sets of electron-nucleus-electrons at 180 degrees versus nucleus vertex by at last one of direction, wavelength, and polarity, as determined by the data set of information. Determination may further include re-examining the model at one or more of a different portions using one or more of changing a distance between a source of the beam of monochromatic physics spin isolated electromagnetic radiation and the substrate over a range configured to irradiate the portions of the substrate at intervals of greater than or equal to about ⅜rₑ, wherein rₑ is the radius of an electron equal to about 2.8179*10⁻¹⁵ m, and/or utilizing a different beam wavelength λ_{EM}, and a different beam direction; a different angle of incidence.

In embodiments, the controller is configured to utilize a series of irradiation pulses and determining variability of photon direction of the series of irradiation pulses, each of the plurality of portions, and compare the resulting spectral line dataset with the predetermined data set to determine the atomic structure of the substrate.

In embodiments, the system for determining an atomic structure of a substrate may be a component of a computer, wherein the atomic structure of at least one portion of the substrate is dependent on an occurrence or non-occurrence for a computer operation. In embodiments, the computer operation comprises a change in electron energy level of an electronic memory of a computer. In embodiments, the substrate comprises a dopant, a crystal lattice, a protein, and/or the like, which undergo a change in atomic structure as part of an occurrence or non-occurrence for a computer operation, which is detected by the system for determining an atomic structure of a substrate according to embodiments disclosed herein. In embodiments, the detection system may include a photomultiplier tube, and electron multiplier tube, and/or the like.

FIG. 5 depicts a spin-isolated monochromatic electromagnetic beam 500 according to embodiments disclosed herein, consisting essentially of a plurality of spin-isolated photons depicted as dashes, wherein the spin-isolated photons are only observable within a plurality of first discrete ranges 502 along a path of the monochromatic electromagnetic beam 500, each of the first discrete ranges 502 is centered at a corresponding distance from a source 504 of the monochromatic electromagnetic beam, wherein essentially no photons are observable within a plurality of second discrete ranges 506 located in-between each of the first discrete ranges 502.

In embodiments, the first discrete ranges 502 wherein the spin-isolated photons are observable, have a length 508 of greater than or equal to about 0.1 nm. In embodiments, the first discrete ranges wherein the spin-isolated photons are observable have a length of less than or equal to about 10 nm. In embodiments, an average length 508 of the first discrete ranges is essentially equal to an average length of the second discrete ranges 510 in which essentially no photons are observable.

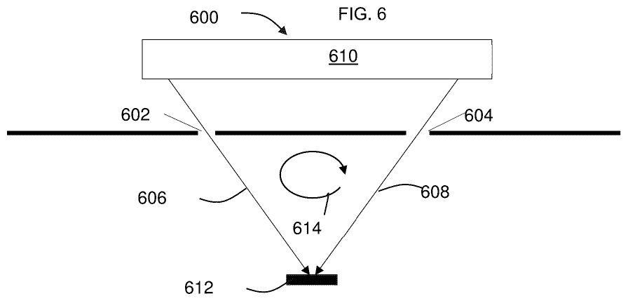

FIG. 6 depicts a modified Aharonov Bohm filter 600 suitable to produce the physics spin isolated electromagnetic energy beam, comprising two slits 602 and 604, through which two monochromatic electromagnetic beams 606 and 608 produced by a source 610, pass through striking a substrate 612, wherein the interference pattern produced by the two monochromatic electromagnetic beams 606 and 608 is a spin isolated electromagnetic energy beam, produced by interaction with a circular magnetic field 614. The direction of the magnetic field 614 is outward from the figure; the inward returning flux is not shown, but is outside the electron paths. The arrow 614 shows the direction of the field which interacts with the two monochromatic electromagnetic beams 606 and 608.

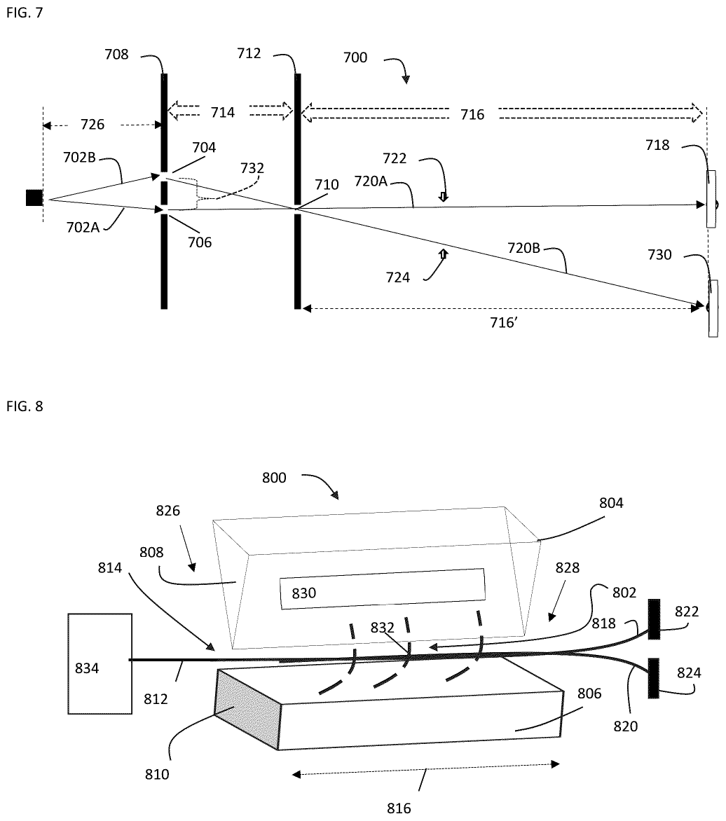

FIG. 7 depicts a modified twin-single slit filter 700 suitable to produce the physics spin isolated electromagnetic energy beam according to embodiments disclosed herein. In embodiments, monochromatic electromagnetic beams 702A and 702B having a wavelength (A) is produced from a source 728 and directed through two coplanar slits 704 and 706 disposed through a first wave barrier 708. A third slit 710 is disposed through a second barrier 712. A spin isolated monochromatic electromagnetic beam 720A produced from the monochromatic electromagnetic beam 702A emanates from third slit 710. Likewise, a spin isolated monochromatic electromagnetic beam 720B produced from the monochromatic electromagnetic beam 702B also emanates from third slit 710 along a different path.

As the monochromatic electromagnetic beams 702A and 702B arrive at the first barrier 708 and pass through the corresponding slits 704 and 706, an interference patter is produced on the second barrier 712 separated from the first barrier 708 by a second distance 714, as is known in the art. However, it is believed that the alternating light band-dark band interference pattern produced on the second barrier 712 is the result of overlapping of the physics spins.

In embodiments, the position of third slit 710 is located within a dark band of the pattern of light and dark bands produced on the second barrier 712. Ostensibly, no photons are present in this region. However, the inventors have discovered that indeed photons are present in this region, they are simply not observable in this region due to interference between different beams, each having a particular spin isolation.

A first target substrate 718 is located at a target distance 716 from the second barrier 712. The spin isolated monochromatic electromagnetic beam 720A has a physics-spin arbitrarily labeled ‘down’ (arrow 722) in this example. The spin isolated monochromatic electromagnetic beam 720B has a physics-spin arbitrarily labeled ‘up’ (arrow 724) in this example.

In embodiments, a plurality of second target substrates 430 (only one of which is shown for simplicity) may be arranged to intersect another spin isolated monochromatic electromagnetic beam 720B at a second target distance 716′, which in embodiments may be located at the first target distance 716.

The spin isolated monochromatic electromagnetic beams 720A and 720B each independently consist essentially of a plurality of spin-isolated photons. These spin-isolated photons are only observable within a plurality of first discrete ranges along a path of the monochromatic electromagnetic beam, each of the first discrete ranges is centered at a corresponding distance from a source of the monochromatic electromagnetic beam. The spin isolated monochromatic electromagnetic beams are further characterized as having a plurality of second discrete ranges located in-between each of the first discrete ranges wherein essentially no photons are observable. Accordingly, only the portion of the target substrate 718 located at the target distance 716 is irradiated with the photons present in the spin isolated monochromatic electromagnetic beam at the corresponding first discrete range. Any portion of the first target substrate 718 which is not present within this first discrete range is not irradiated with the photons present in the spin isolated monochromatic electromagnetic beam. Accordingly, in embodiments, the target distance 716 may be controlled and/or modified to provide spatial resolution of irradiation or other types of activation on or of the target substrate by the spin isolated monochromatic electromagnetic beam.

In embodiments, the target distance 716 is selected, modified, controlled and/or configured such that photons of the spin isolated monochromatic electromagnetic beam 720A irradiate, activate, and/or interact with the portion of the first target substrate to be irradiated, allowing the intended result of that irradiation e.g., photocatalytic curing of a mask, only in the region irradiated.

It is noted that the prior art use of the terms ‘up’ and ‘down’ to describe spin is misleading. An ‘up’ spin of a spin isolated monochromatic electromagnetic beam is more completely described as having an inner-clockwise/outer-counterclockwise spin, and a ‘down’ spin of a spin isolated monochromatic electromagnetic beam is more completely described as inner-counterclockwise/outer-clockwise with the center as the same node. Accordingly, consistent with current understanding, photons and EM waves possess dual wave characteristics (See Vigen, A. (Dec. 24, 2023). The Nature and Causation of Light; Amazon-ASIN B0CQXD14QN).

Each spin isolated monochromatic electromagnetic beam arriving at a barrier will show a pattern of alternating light bands where photons are observable and dark bands in which intermediate positions are interfering where photons are not observable and thus no activation of a substrate brought about by irradiation with photons will occur.

It is theorized that spin isolated monochromatic electromagnetic beams are produced via spin aggregation of the photons, in which both hemispheres of the subatomic particle, in this case the photon, move in the same direction and thus there is no change in the energy-level associated with the photons present in the spin isolated monochromatic electromagnetic beam.

In embodiments, the portion of the substrate to be activated or irradiated by the photons of the spin isolated monochromatic electromagnetic beam is located in an activation position, which is coincident with an entanglement node of the spin isolated monochromatic electromagnetic beam, where the spin of each photon present is inside one direction and outside the other, resulting in a change in the energy-level for a particular photon, or other subatomic particle i.e., protons and electrons. The rotation energy being the transposition rate of those poles defined by the subatomic particle's axis, with the resulting change in the B-field for perpendicular magnetism linear acceleration according to relationships known in the art.

FIG. 8 depicts a modified Stern Gerlach magnetic spin isolation filter 800 suitable to produce the physics spin isolated electromagnetic energy beam, comprising a non-uniform magnetic field 802 disposed between an inlet 826 and an outlet 828 of the magnetic spin isolation filter 800. In embodiments, magnetic spin isolation filter 800 includes a first magnet 804 and a second magnet 806, the first magnet 804 having a first cross section 808 oriented perpendicular to a path of the monochromatic electromagnetic beam 812; the second magnet 806 having a second cross section 810 oriented perpendicular to the path of the monochromatic electromagnetic beam 812 produced by a source 834, which is different from the first cross section 808, such that the opening 814 between the two magnets 804 and 806 has a non-uniform magnetic field having a magnetic field strength 832 and length 816 in the direction of the path of the monochromatic electromagnetic beam 812 sufficient to separate the monochromatic electromagnetic beam 812 into a pair of spin-isolated electromagnetic energy beams 818 and 820. As shown in FIG. 8, at least one of the spin-isolated electromagnetic energy beams 818 and 820 emanating from the outlet 828 interact with a target substrate 822 and 824 to affect actuation or a change of a portion of the target substrate, as described above.

In embodiments, the first magnet 804, the second magnet 806, or both are, or include one or more electromagnetic elements 830 configurable to adjust the magnetic field strength 832 one or more points along the path of the monochromatic electromagnetic beam 812.

While the foregoing is directed to embodiments of the present disclosure, other and further embodiments of the disclosure may be devised without departing from the basic scope thereof.

1. A method to determine an atomic structure of a substrate, comprising:
irradiating a plurality of portions of the substrate with a beam consisting essentially monochromatic physics spin isolated electromagnetic radiation having a beam wavelength λ_{EM}, oriented in a beam direction, the beam contacting the substrate at an angle of incidence relative to the substrate, for a period of time sufficient to produce an emission of photons from each of the plurality of portions forming one or more emission spectral lines corresponding to a particular portion of the substrate, wherein the emission photons emanate from the substrate at an emission angle which is different from the angle of incidence and are detected by a detection system;
analyzing the emission spectral lines produced by the photons from each of the plurality of portions to produce an emission spectral line dataset, comprising:
an emission angle of the emission photons which formed the corresponding spectral line relative to the beam direction;
a wavelength of each corresponding spectral line λ_{sub}, and/or
a polarity of the photons forming the corresponding spectral line; and
determining the atomic structure of the substrate based at least in part on the spectral line dataset.

2. The method of claim 1, wherein the determining of the atomic structure comprises determining an arrangement of subatomic particles and the subatomic particles present at each of the plurality of portions based at least in part on the corresponding spectral line dataset, wherein a distance between a nucleus and an electron of an atom of element E which produced the emission spectral line is determined according to formula (I):
$${λ}_{sub} = {R}_{E , ⊖ \#} ( 1 / {( {N}_{1} )}^{2} - 1 / {( {N}_{2} )}^{2} ) ; \qquad ( I )$$
wherein:
λ_{sub} is a wavelength of the emission spectral line;
θ# is an integer second quantum number of the subshell of the electron of the atom which produced the emission spectral line;
R_{E,θ#} is a Rydberg constant for the subshell of the electron of the atom of element E which produced the emission spectral line;
Nₓ is an integer subset energy level of the element E of the atom which produced the emission spectral line, starting at x=1, determined by formula (II)
$${N}_{x + 1} = {N}_{x} + 1 ; \qquad ( II )$$
wherein each N and x are determined independently, as integers greater than or equal to 1; and
wherein R_{E,θ#} is determined by formula (III):
$${R}_{E , ⊖ \#} = 6 {r}_{e} ( {( {D}_{eN} / ( 6 {r}_{e} ) )}^{2} - 1 ) ; \qquad ( III )$$
wherein:
rₑ is the radius of an electron; and
D_{eN} is the distance between the nucleus and the electron of the atom of the element E which produced the emission spectral line.

3. The method of claim 2, wherein the determining of the atomic structure further comprises determining an emission angle between an xtrastatic axis of the nucleus and the electron of the atom of the substrate which produced the emission spectral line, wherein the nucleus is a vertex of the angle, according to formula (IV):
$$cos ( {⊖}_{emission} ) = \frac{1}{( 2 * ( r \# ) - 1 )} \qquad ( IV )$$
wherein
θₑₘᵢₛₛᵢₒₙ is the emission angle in radians, ±0.1 radians, of the electron relative to the xtrastatic axis of the nucleus and the electron of the atom of the substrate which produced the emission spectral line, with the nucleus as the vertex for the atomic structure of the substrate;
θ# is an integer second quantum number of the subshell of the electron of the atom which produced the emission spectral line; and
r# is the integer first quantum number of the shell of the electron of the atom which produced the emission spectral line.

4. The method of claim 3, wherein the determining of the atomic structure further comprises determining an electron arrangement of the atom of the substrate which produced the emission spectral line;
wherein a direction of covalent bonding between the atom of the substrate which produced the emission spectral line and another atom present within the substrate is based at least in part on a predetermined data set comprising a plurality of predetermined distances between a nucleus and an electron of an atom of element E, and a plurality of predetermined emission angles between bonding electrons and a corresponding nucleus of an atom of element E, wherein:
the predetermined data set includes only full subshells sets; and
the subshell sets are selected from one or more of:
Subshell-s having 2 electrons;
Subshell-p having 6 electrons;
Subshell-d having 10 electrons;
Subshell-f having 14 electrons; and/or
equatorial (π radian) sets having 3, 5, or 7 electrons each.

5. The method of claim 1, further comprising changing a distance between a source of the beam of monochromatic physics spin isolated electromagnetic radiation and the substrate over a range configured to irradiate the portions of the substrate at intervals of greater than or equal to about ⅜ rₑ, wherein rₑ is the radius of an electron equal to about 2.8179*10⁻¹⁵ m.

6. The method of claim 1, wherein the irradiating of the plurality of portions of the substrate comprises a plurality of irradiations of the same substrate, each utilizing a different beam wavelength λ_{EM};
a different beam direction;
a different angle of incidence;
or a combination thereof.

7. The method of claim 1, wherein the substrate comprises a protein sequence.

8. The method of claim 1, wherein the substrate comprises a semi-conductor substrate.

9. The method of claim 1, wherein the atomic structure of at least one portion of the substrate is dependent on an occurrence or non-occurrence for a computer operation.

10. The method of claim 9, wherein the computer operation comprises a change in electron energy level of an electronic memory of a computer.

11. A system for determining an atomic structure of a substrate, comprising:
a source of a beam of monochromatic physics spin isolated electromagnetic radiation having a beam wavelength λ_{EM},
an irradiation system configured to irradiate a plurality of portions of a substrate with the beam of monochromatic physics spin isolated electromagnetic radiation in a beam direction such that the beam contacts the substrate at an angle of incidence relative to the substrate for a period of time sufficient to produce an emission of photons from each of the plurality of portions forming one or more emission spectral lines corresponding to a particular portion of the substrate,
an analysis system configured to analyze the emission photons emanating from the substrate at an emission angle which is different from the angle of incidence;
the analyzing system configured to determine the emission spectral lines produced by the photons from each of the plurality of portions to produce an emission spectral line dataset comprising:
an emission angle of the emission photons which formed the corresponding spectral line relative to the beam direction;
a wavelength of each corresponding spectral line λ_{sub}, and/or
a polarity of the photons forming the corresponding spectral line; and
determining the atomic structure of the substrate based at least in part on the spectral line dataset.

12. The system of claim 11, further configured to change a distance between the source of the beam of monochromatic physics spin isolated electromagnetic radiation and the substrate over a range configured to irradiate the portions of the substrate at intervals of greater than or equal to about ⅜ rₑ, wherein rₑ is the radius of an electron equal to about 2.8179*10⁻¹⁵ m.

13. The system of claim 11, further configured to change the beam wavelength λ_{EM};
the beam direction;
the angle of incidence;
or a combination thereof.

14. The system of claim 13, configured to conduct a plurality of irradiations on the same substrate, each utilizing a different beam wavelength λ_{EM};
a different beam direction;
a different angle of incidence;
or a combination thereof.

15. The system of claim 11, wherein the atomic structure of at least one portion of the substrate is dependent on an occurrence or non-occurrence for a computer operation.

16. The system of claim 15, wherein the computer operation comprises a change in electron energy level of an electronic memory of a computer.
